(19) United States
(12) Patent Application Publication
(10) Pub. No.: US 2009/0018017 A1
(43) Pub. Date: Jan. 15, 2009

(54) Composition for inhibiting germination of seeds and production method thereof

(76) Inventors: Ha-Ju Hwang, Gwangju Metropoliatn City (KR); Sun-Yong Jung, Gwangju Metropolitan City (KR); Gyeong-Taek (Kyung-Taek) Oh, Gwangju Metropolitan City (KR)

Correspondence Address: WANG, HARTMANN & GIBBS, 1301 DOVE STREET, SUITE 1050, NEWPORT BEACH, CA, 92660, US

(21) Appl. No.: 12/074,078

(22) Filed: Feb. 28, 2008

(30) Foreign Application Priority Data
Jul. 10, 2007 (KR) 10-2007-0069342

(51) Int. Cl. A01N 63/04; C12N 1/20

(52) U.S. Cl. 504/117; 435/253.6

(57) ABSTRACT

This invention relates to a composition including Pseudomonas sp. strain for inhibiting germination of seeds and production method thereof. By sprinkling the composition containing germination inhibitor on seeds, germination of seeds can be suppressed environment-friendly without any social problems including environment, hygiene, and health which can be caused by using chemical products.

### FIELD OF THE INVENTION

This invention relates to a composition for inhibiting germination of plant seeds and production method thereof. In particular, a composition includes Pseudomonas aeruginosa, which is a microorganism, for inhibiting germination of plant seeds and production method thereof.

### BACKGROUND OF THE INVENTION

In general, plant seeds begin germination by activation of α-amylase in appropriate temperature and moisture. Germinated seeds are without, or have a significantly reduced value as the material for processing into bread, beer, or beverages.

In order to store seeds for long time without germination, a number of methods have been used, including physical method which controls the environment of storage such as temperature, humidity, and illumination, MH-30, antiseptics, chemical preservation method using chemicals such as chloride, and radiation treatment using the gamma rays from ⁶⁰Co or ¹³⁷ Cs.

However, physical preservation methods have the demerit of large investment in storage facilities, though the method can achieve good result for the storage of rice, wheat, barley, etc., and the radiation method has the problem of completely destructing the germinating power of seeds in addition to large investment in facilities.

Chemical preservation methods have the demerit of toxicity of chemicals for articles of food, though the seeds can be used for breeding, and the method requires additional chemical treatment to promote germination. Furthermore, use of chemicals causes a number of social and environmental problems in addition to human health and mutation of seeds.

### SUMMARY OF THE INVENTION

To this end, this invention was developed to solve the above mentioned problems. The main object of this invention is to present a composition that can inhibit the germination of seeds environment-friendly without social problems in environment, hygiene, and health, which may be caused by the use of chemicals.

Another object of the present invention is to provide a composition for inhibiting germination of seeds, which contains the culture solution obtained from C-culture media which cultures Pseudomonas aeruginosa (Pseuclomonas sp. strain F-721 which secretes germination inhibitor, registered in the Gene Bank, Life Science Research Institute, KAIST).

In addition, another object of the present invention is to provide a method for producing a composition which has enhanced capability of inhibiting germinating power of seeds by controlling the temperature and pH of the C-culture media which cultures Pseudomonas aeruginosa.

In addition, another object of the present invention is to provide a method for producing a composition which has enhanced capability of inhibiting germinating power of seeds by adding gibberelling in the C-culture media which cultures Pseudomonas aeruginosa.

In addition, another object of the present invention is to provide a method for suppressing the germination of seeds in storage or distribution by using the said seed germination inhibitor.

### BRIEF DESCRIPTION OF THE DRAWINGS

FIG. 1 shows the seed germination inhibition effect of the composition which includes the seed germination inhibition substance produced with Pseudomonas aeruginosa;

FIG. 2 shows the germination inhibition efficiency of composition in accordance with the present invention, according to the culturing temperature of Pseudomonas aeruginosa;

FIG. 3 shows the germination inhibition efficiency of composition in accordance with the present invention, according to the culturing pH of Pseudomonas aeruginosa; and,

FIG. 4 shows the germination inhibition efficiency of composition in accordance with the present invention, according to the plant hormone.

### DETAILED DESCRIPTION OF THE INVENTION

In one embodiment, the objects of the present inventions described herein above can be achieved by conducting the following procedures. The microorganisms in soil were taken and cultured to identify and select the strain that secretes seed germination inhibitor.

The isolation procedures of the said strain of the present invention includes following steps;

(1) obtain soil and smear it on the gelatin culture media;
(2) isolation of the green colored strains into single colony from the gelatin culture media;
(3) culture each strain, by shaking, in Luria Bertani(LB) liquid culture media, collect the strain and suspend in sterilized water of the same volume;
(4) sprinkling of the said suspension on seeds;
(5) selecting strain by examining the germination of the seeds,

The constitution of the present invention is described in detail hereinbelow; In the step (1), suspend the soil or plant taken to isolate the microorganisms, which have the capability of suppressing germination of seeds, in sterilized water, and smear it on LB gelatin culture media. In this step (2), isolate the strains, which show green color on the gelatin culture media into single colony.

In the step (3), culture each strain by shaking in LB liquid culture media at 28° C., at the rotation speed of 200 rpm for 24 hours, collect the micro-organisms by centrifugal isolation method, and suspend it in sterilized water of the same volume.

In the step (4), sprinkle the said suspension on plant seeds.

in the step (5), observe the germination of the seeds for at least 1 week after step (4) to screen the strain that has inhibiting power against the germination of seeds.

Through above described steps, a new microorganism was isolated from soil and identified. The isolated strain was tested with Bergey's Manual of Systematic Bacteriology, the strain was identified to be Pseudomonas aeruginosa.

The shape and size of the said microorganism of the present invention was observed with optical microscope (Olympus BS51TR-3200, Japan) and FE-SEM(HITACHI-S4500II), and the results are listed in Table 1.

TABLE 1

| Classification | Characteristics |
|---|---|
| Gram strain | — |
| Shape | Bacillus |
| Width | 0.5-0.6 (μm) |
| Length | 1.8-2.60 (μm) |

The Physicochemical inspection of the Pseudomonas aeruginosa in the present invention was conducted with API 20NE test strip(bioMerieux Co, France), and the results are listed in Table 2 and 3.

TABLE 2

| Classification | Characteristics |
|---|---|
| Catalase(catalase production) | + |
| Oxidase(cytochrome oxidase production) | + |
| Potassium nitrate(reduction of nitrate) | + |
| Use of tryptopahn(produce indole) | − |
| Use of glucose(oxidation) | − |
| Use of arginine(produce arginine dihydrolase) | + |
| Use of urea(produce urase) | − |
| Use of esculin(esculin hydrolysis) | − |
| Use of gelatin(gelatin hydrolysis) | − |

TABLE 3

| Classification | Characteristics |
|---|---|
| Manidol | − |
| Inosidol | − |
| Ediphate | + |
| Arabinose | − |
| Nitrate production | + |
| Nitrogen production | + |
| Oxidation | + |

The conventional seed preservation methods including physical, chemical, and radiation exposure have caused a number of problems including large investments, chemical residue in seed, and social and environmental problems related with the use of radiation.

In contrast, the method of the present invention, which suppresses seed germination using Pseudomonas aeruginosa can inhibit seed germination environment-friendly with less cost without diverse problems in environment, hygiene, and health caused by using chemicals.

Hereinafter, the contents of the present invention is described in detail by an example, however, the scope of the claims of the present invention is not limited to the description below.

Isolation and Identification of Pseudomonas aeruginosa Strain

A number of the microorganisms which can decompose oil and VOCs (Volatile Organic Compounds) were investigated. The Pseudomonas aeruginosa of the present invention was one of the microorganisms which have the inhibition power against seed germination. Isolated strain was identified by Bergey's Manual of Systematic Bacteriology method.

The microorganisms which produce seed germination inhibitor were sampled from the nature and isolated by using the C-culture media which contain the ingredients listed in Table 4, per 1 liter of distilled water. PH was set at 7.0, glucose was used as the substrate. Isolated strain was mixed with glycerol and stored at −70° C.

TABLE 4

| Ingredient | Concentration |
|---|---|
| (NH₄)S0₄ | 5.0 g |
| KH₂PO₄ | 1.0 g |
| K₂HP0₄ | 2.0 g |
| MgSO•7H₂0 | 0.2 g |
| NaCl | 2.0 g |
| CaCl₂ | 10 mg |
| FeSO₄•7H₂0 | 10 mg |
| Yeast extract | 0.2 g |
| Trace element solution | 2.0 ml |
| PH | 7.0 |
| Distilled water | 1 L |

Culturing of Pseudomonas Aeruginosa Strain

A) Culturing of Germination Inhibitor Strain 0.1% (w/v) of glucose, as the single carbon source, was added into C-culture media, sterilized, and added with the Pseudomonas aeruginosa of the present invention by 1%, and cultured in 35° C., 200 rpm reciprocal shaker for 24 hours. The cultured strain was isolated centrifugally at 10,000 rpm for 15 minutes into pellets and supernatant. Pellets were suspended in sterilized water, and the supernatant was filtered with 0.45 μm sterilized syringe filter to eliminate pellets completely.

B) Examination of the Inductivity of the Germination Inhibiting Product 0.1% (w/v) of glucose, as the single carbon source, was added into C-culture media, sterilized, and added with the Pseudomonas aeruginosa in accordance with the present invention by 1%. 200 g of barley was sterilized with 70% alcohol for 1-5 minutes then washed with sterilized distilled water several times in clean bench, added into the culture media, and cultured in a 200 rpm reciprocal shake at 35° C. for 24 hours. Culture strain was centrifuged at 10,000 rpm for 15 minutes to isolate pellets and supernatant. the pellets were suspended in sterilized water, and the supernatant was filtered with 0.45 μm sterilized syringe filter to eliminate residual pellets.

The inductivity of the germination inhibiting product was examined by comparing with the experiment A).

<Exemplary Implementation 1> Examination of the Inhibition Efficiency Against Seed Germination

### A) Seed Germination Testing Method

In a sterilized petri dish placed with filter paper, the strain and filtered fluid obtained by centrifugal isolation were diluted and added by step. Various plant seeds were placed on the filter paper by 20 each, and cultured for 5 days at room temperature to test the germination power by comparing with the control group. The control group was prepared by placing filter paper in sterilized petri dishes filled with sterilized water, laid with various plant seeds to conduct the experiment in the same manner as for the test group. The seeds tested in the exemplary implementation were radish, Chinese cabbage, lettuce, barley, and sesame. The efficiency of inhibition was calculated by comparing the length of the roots of the test group and control group.

B) Comparison Analysis of Germination Inhibition Efficiency

The efficiency of germination inhibition of Pseudomonas aeruginosa on various plant seeds in accordance with the experiment described above was shown in Table 5 below and FIG. 1.

The inhibition efficiencies were calculated with the length of roots. All the tests were conducted at room temperature (approx. 20° C.).

TABLE 5

| | | | | 400% | 800% |
|---|---|---|---|---|---|
| | | No dilution | 200% dilution | dilution | dilution |
| Pseudomonas | | Efficiency | Efficiency | Efficiency | Efficiency |
| aeruginosa | | (%) | (%) | (%) | (%) |
| Radish | (1)P | 51 | 70 | 68 | 45 |
| | (2)P | 55 | 0 | 0 | 0 |
| | (1)F | 98 | 98 | 70 | 21 |
| | (2)F | 98 | 25 | 25 | 26 |
| Chinese | (1)P | 56 | 54 | 42 | 0 |
| Cabbage | (2)P | 10 | 44 | 0 | 0 |
| | (1)F | 100 | 98 | 98 | 98 |
| | (2)F | 65 | 40 | 38 | 4 |
| Lettuce | (1)P | 88 | 81 | 5 | 25 |
| | (2)P | 52 | 25 | 25 | 0 |
| | (1)P | 100 | 100 | 100 | 100 |
| | (2)P | 99 | 99 | 76 | 58 |
| Barley | (1)P | 0 | 53 | 21 | D |
| | (2)P | 81 | 34 | 0 | 32 |
| | (1)F | 100 | 100 | 100 | 6 |
| | (2)F | 81 | 47 | 60 | 0 |
| Sesame | (1)P | 37 | 66 | 58 | 40 |
| | (2)P | 47 | 36 | 40 | 43 |
| | (1)F | 100 | 61 | 81 | 81 |
| | (2)F | 81 | 64 | 60 | 60 |

In Table 5 above, (1)P refers to the pellet obtained from the Pseudomonas aeruginosa and sterilized barley in C-culture media(glucose 0.1%), cultured in incubator for 24 hours at 35° C., 200 rpm, and centrifuged at 10,000 rpm for 15 minutes, (2)P refers to the pellet obtained from the Pseudomonas aeruginosa in C-culture media(glucose 0.1%), cultured in incubator for 24 hors at 35° C., 200 rpm, and centrifuged at 10,000 rpm for 15 minutes, (1)F is the filtrate obtained by filtering the supernatant obtained from the Pseudomonas aeruginosa and sterilized barley in C-culture media(glucose 0.1%), cultured in incubator for 24 hours at 35° C., 200 rpm, and centrifuged at 10000 rpm for 15 minutes, (2)F is the filtrate obtained by filtering the supernatant with filter paper (0.45 μm sterilized syringe filter) obtained from the Pseudomonas aeruginosa in C-culture media(glucose 0.1%), cultured in incubator for 24 hours at 35° C., 200 rpm, and centrifuged at 10,000 rpm for 15 minutes.

The germination inhibition effect was higher in the specimen added with filtrate than those added with Pseudomonas aeruginosa directly. In the specimens added with Pseudomonas aeruginosa directly, the germination inhibition effects showed inconsistency according to the degree of dilution. This shows that Pseudomonas aeruginosa secretes a certain substance, which has the germination inhibition power. Since the optimal culturing temperature of Pseudomonas aeruginosa is 35° C., the inhibition power became weaker due to the temperature, but the secretion can give optimal inhibition effect regardless of the temperature.

Considering the test results of the filtrate, the filtrate obtained by adding barley together with Pseudomonas aeruginosa, cultured, centrifuged, and filtered showed significantly higher germination inhibition effect than the filtrate obtained by culturing Pseudomonas aeruginosa only. Therefore, it could be seen that the germination inhibitor secreted y Pseudomonas aeruginosa is inductive. In addition, highly diluted use also gives high inhibition effect, which is a merit for economy.

<Exemplary Implementation 2> Search for the Culturing Conditions for Pseudomonas aeruginosa

With the isolated Pseudomonas aeruginosa strain, optimal culturing conditions were investigated using glucose as the source of carbon. Temperatures wee set at 25° C., 30° C., and, 35° C., pH was set at 6.0˜9.0, and glucose concentration was set at 0.1˜15.0% (w/v) to test the optimal culturing conditions. To measure the growth of the selected strain, samples were taken at every 2 hours from the culture fluid and absorbance at 600 nm with spectrophotometer and CFUs (colony forming-units) were tested. The concentration of the glucose used as the carbon source was measured by DNS method and glucose measuring reagent method.

The optimal culturing temperature and pH conditions of Pseudomonas aeruginosa are shown in FIG. 2 and FIG. 3 respectively.

Considering that the Pseudomonas aeruginosa strain cultured at the temperature of 25° C.˜35° C. showed highest inhibition power, which means that the strain is cultured best the test temperature.

In addition, seeing that the germination inhibition effect of the culture fluid obtained at pH 9.0 far superceded the fluid obtained by other pH conditions, even after time, it could be known that the best pH condition for culture is 9.0.

<Exemplary Implementation3> Induction of Seed Germination Inhibition Substance from Pseudomonas aeruginosa
To examine the inductivity of the germination inhibition substance, the said strain was cultured by adding gibberellin (GA), indol acet acid (IAA), and benzylaminopurin (BAP), 1 mM each, were added in addition to glucose, In C-culture media. Pellet and supernatant were isolated by centrifuging the culture fluid for 10 minutes at 10,000 rpm. The pellet was suspended in sterilized water and the supernatant was filtered with 0.45 μm sterilized syringe filter to eliminate pellet, and used in the test for the Exemplary Implementation 1.

FIG. 4 shows the results of the Exemplary Implementation 3. In the induction test of germination inhibition substances using plant hormone, gibberellin (GA) showed larger inductivity of germination inhibition substance than the IAA of auxins series and BAP of cytokinins.

Therefore, the seed germination inhibition substance can be induced more from Pseudomonas aeruginosa by adding gibberellin in the C-culture media.

What is claimed is:

1. A method of suppressing the germination of plant seeds, comprising the steps of;
securing a culture fluid obtained from Pseudomonas aeruginosa cultured in C-culture media which contains (NH₄)SO₄ 5.0 G, KH₂PO₄ 1.0 g, K₂HPO₄ 2.0 g, MgSO₄.7H₂0 0.2 g, NaCl 2.0 g, CaCl₂ 10 mg, FeS0₄. 7H₂0 10 mg, and yeast extract 0.2 g per 1 liter of distilled water; and
applying said culture fluid to seeds.

2. The method of suppressing the germination of plant seeds in accordance with claim 1, wherein the Pseudomonas aeruginosa is cultured in C-culture media, at pH 9.0 and at 25° C.-35° C.

3. The method suppressing the germination of plant seeds in claim 2, wherein the method includes adding gibberellin in the said C-culture media.

4. The method of suppressing the germination of seeds by applying the seed germination inhibition composition which is produced in accordance with the claim 2 or claim 3.

5. A culture fluid for cultivating Pseudomonas aeruginosa comprising:
C-culture media containing (NH₄)SO₄ 5.0 G, KH₂PO₄ 1.0 g, K₂HPO₄ 2.0 g, MgSO₄. 7H₂0 0.2 g, NaCl 2.0 g, CaCl₂ 10 mg, FeS0₄.7H₂0 10 mg, and yeast extract 0.2 g per 1 liter of distilled water.

6. The culture fluid of claim 5 wherein the culture fluid is at a 9.0 Ph and at 25° C.-35° C.

7. The culture fluid of claim 5 further containing gibberellin in the said C-culture media.

8. A product for suppressing the germination of plant seeds made by the process comprising the steps:
producing a culture fluid from Pseudomonas aeruginosa cultured in C-culture media which contains (NH₄)SO₄ 5.0 G, KH₂PO₄ 1.0 g, K₂HPO₄ 2.0 g, MgSO₄.7H₂0 0.2 g, NaCl 2.0 g, CaCl₂ 10 mg, FeS0₄.7H₂0 10 mg, and yeast extract 0.2 g per 1 liter of distilled water; and
extracting the culture fluid.

9. A method of suppressing germination of plant seeds comprising applying the culture fluid from Pseudomonas aeruginosa to the plant seeds.
